(19) United States
(12) Patent Application Publication
(10) Pub. No.: US 2012/0323286 A1
(43) Pub. Date: Dec. 20, 2012

(54) DEVICE FOR THE DISTRACTION OF A BONE SEGMENT

(76) Inventor: CHRISTIAN KRENKEL, SALZBURG (AT)

(21) Appl. No.: 13/230,020

(22) Filed: Sep. 12, 2011

(30) Foreign Application Priority Data
Jun. 17, 2011 (EP) 11004968.1

(51) Int. Cl. A61B 17/84

(52) U.S. Cl. 606/328

(57) ABSTRACT

A device for distraction of a bone segment from local bone, or endo-distractor. The device has a rotatable, smooth shaft adjoined by a straight anchoring screw. During the unscrewing of the anchoring screw a carrier piece transfers the axial movement but not the rotation to the bone segment. The carrier piece for the bone segment is a pressure plate which is formed with an open slit and which, by being pushed orthogonally, with respect to the longitudinal direction of the shaft, onto the shaft, can be secured on the latter in the axial direction.

### CROSS-REFERENCE TO RELATED APPLICATION

This application claims the priority, under 35 U.S.C.§119, of European patent application EP 11004968.1, filed Jun. 17, 2011; the prior application is herewith incorporated by reference in its entirety.

### BACKGROUND OF THE INVENTION

### Field of the Invention

The invention relates to a device for distraction of a bone segment from local bone, also referred to as an endo-distractor. The device has a rotatable, smooth shaft adjoined by a straight anchoring screw, during the unscrewing of which a carrier piece transfers the axial movement but not the rotational movement to the bone segment.

A device of this kind that has proven useful in practice is known from U.S. Pat. No. 6,666,869 B2 and its counterpart European patent EP 1 259 177 B1.

There, there is provided a carrier piece, which presses the bone segment away from the local bone. The carrier piece consists of a sleeve which surrounds the shaft and which, during the unscrewing of the anchoring screw, is acted upon by an annular flange that surrounds the shaft. To be able to transfer the corresponding pressure to the bone segment, the sleeve is screwed into the latter. After the procedure has been completed, the sleeve must accordingly be removed from the bone segment by being unscrewed.

### SUMMARY OF THE INVENTION

It is an object of the invention to provide a novel endo-distractor which overcomes the above-mentioned disadvantage of the heretofore-known device which is further improved over the heretofore-known devices and methods of this general type.

As the objective and fundamental mode of operation of the novel device does not differ from the device described in the above-noted prior disclosures, the prior specifications (U.S. Pat. No. 6,666,869 B2, EP 1 259 177 B1, and progeny) are therefore incorporated by reference in their entirety.

With the foregoing and other objects in view there is provided, in accordance with the invention, a device for distracting a bone segment from a local bone, the device comprising:

a rotatable, substantially smooth shaft;

a straight anchoring screw adjoining said shaft;

a pressure plate forming a carrier piece for the bone segment, said pressure plate transferring an axial movement but not a rotational movement to the bone segment during an unscrewing of said anchoring screw;

said pressure plate having an open slit formed therein, enabling said pressure plate to be pushed in orthogonally with respect to a longitudinal direction of the shaft, for securing said pressure plate in an axial direction on said shaft.

In other words, the invention avoids the stated disadvantage by virtue of the fact that the carrier piece for the bone segment is a pressure plate which is formed with an open slit and which, by being pushed in orthogonally with respect to the longitudinal direction of the shaft (i.e., sideways, transversely), can be secured on the latter in the axial direction.

A particular advantage of the invention is that it completely dispenses with the need to use a sleeve surrounding the shaft, which also means that the problem of the sealing between sleeve and shaft does not arise and, therefore, the structure is considerably simpler compared to the prior art. The only requirement for this is that the diameter of the shaft is not smaller than that of the anchoring screw. This feature is necessary to ensure that, when the procedure is complete, the anchoring screw can be removed through the bore enclosing the shaft.

In principle, the pressure plate according to the invention could be axially secured by means of flanges, split pins or the like surrounding the shaft. If the aim is to dispense with the use of a sleeve surrounding the shaft, provision must then once again be made that this flange or split pin can be inserted orthogonally with respect to the longitudinal direction of the shaft, after the anchoring screw has been inserted, and can be removed again orthogonally prior to the removal. Such a flange or split pin is preferably avoided altogether by securing the pressure plate on the shaft using a circumferential groove into which the slit of the pressure plate engages.

Other features which are considered as characteristic for the invention are set forth in the appended claims.

Although the invention is illustrated and described herein as embodied in a device for distraction of a bone segment, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a diagrammatic representation of an illustrative embodiment of the device according to the invention during use on an upper jaw;

FIG. 2 is a longitudinal section through a slightly modified exemplary embodiment of the device according to FIG. 1; and

FIGS. 3A to 3C show diagrams of three variants of the pressure plate 6 that forms the core of the invention, in conjunction with the groove 9 surrounding the shaft 1.

### DETAILED DESCRIPTION OF THE INVENTION

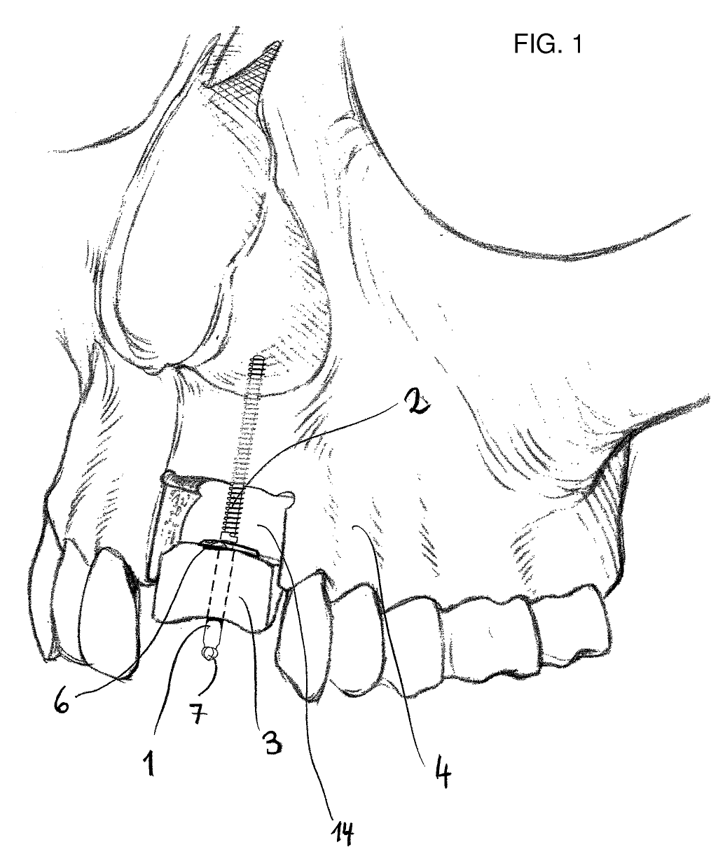

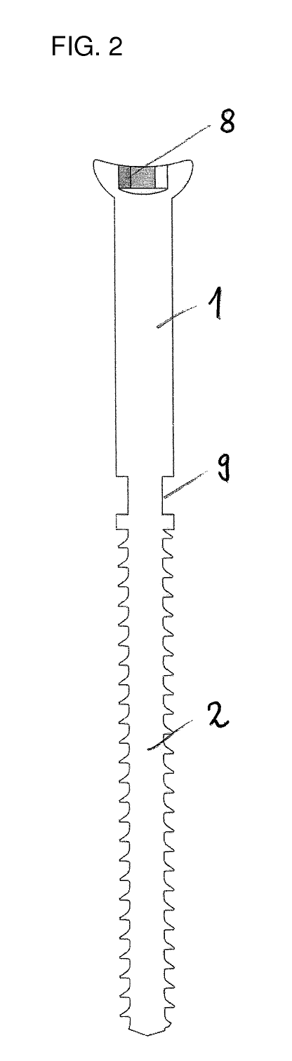

Referring now to the figures of the drawing in detail and first, particularly, to FIG. 1 thereof, there is shown a device according to the invention that serves, similar to the prior art, to build up new tissue in an osteotomy gap between a bone segment 3 and local bone 4. For this purpose, by rotation of the shaft 1 and of the anchoring screw 2, this gap is brought from a starting position, in which only a small osteotomy gap is present, to the position shown, with new tissue gradually forming in the gap 14. The rotation of the shaft 1, and of the anchoring screw 2 forming an integral continuation thereof, is effected by the patient himself or herself, by means of the knurled screw head 7. This is easier than rotation by means of a hexagon socket, as is provided according to FIG. 2.

A rotation of the bone segment 3 is prevented by the side walls of the recess in the bone 4, by the taut continuous gum on the alveolar ridge and by the newly formed tissue. In the case of large bone segments, which are distracted by two parallel devices, a rotation movement is in any event prevented by the respective other unit.

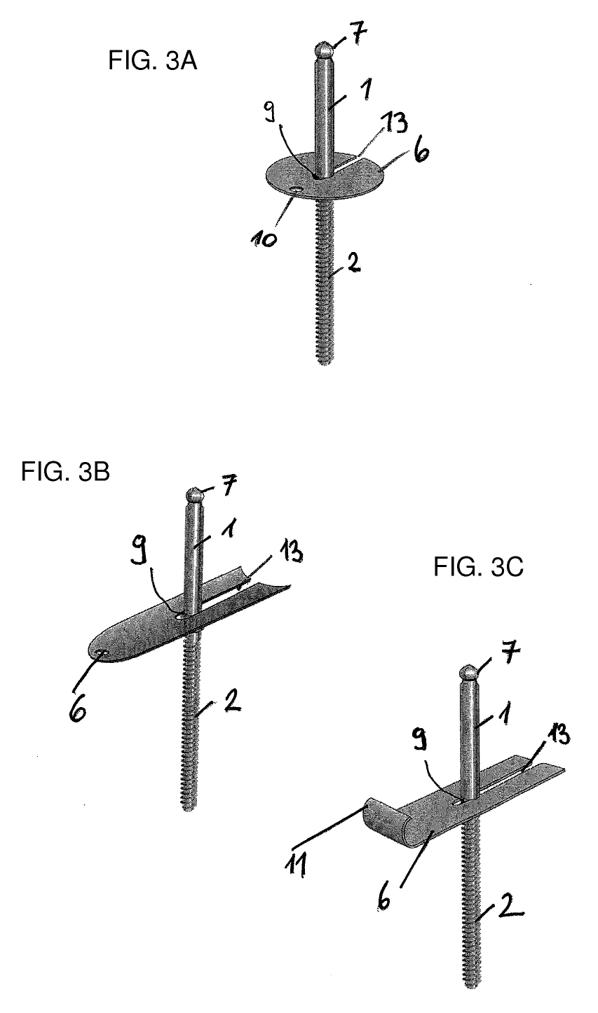

To transfer the lengthwise movement of the anchoring screw 2 to the bone segment 3, a pressure plate 6 is used according to the invention. As can be seen from FIG. 2, the pressure plate 6 is secured on the shaft 1 by means of a circumferential groove 9 on the shaft 1. As can be seen from FIG. 3, the outer shape of the pressure plate 6 can vary considerably. A primarily important aspect is the open slit 13, of which the width corresponds to the diameter of the shaft in the area of the groove 9, such that the pressure plate 6 bearing on the bone segment 3 follows along with the axial movement of the shaft 1 when the anchoring screw 2 in the base bone is unscrewed by way of the screw head 7.

FIG. 3A shows the basic shape of the pressure plate 6 which, at the start of the treatment, is implanted orthogonally after the anchoring screw 2 has been inserted. To make it easier to remove the pressure plate 6 on completion of the procedure, the pressure plate 6 is provided with a bore. The pressure plate 6 can have an elongate design, as is shown in FIG. 3B, if this is needed to guide it as far as the edge of the bone segment 3. However, an elongate pressure plate 6 of this kind should be reliably prevented from following along with the rotation of the anchoring screw 2. For this purpose, it can be provided, for example as is shown in FIG. 3C, with an upwardly curved edge 11, which holds it securely on the bone segment 3.

After completion of the treatment, it may not be necessary to remove the pressure plate 6 if it is made of resorbable material.

1. A device for distracting a bone segment from a local bone, the device comprising:
a substantially smooth, rotatable shaft;
a straight anchoring screw adjoining said shaft;
a pressure plate forming a carrier piece for the bone segment, said pressure plate transferring an axial movement but not a rotational movement to the bone segment during an unscrewing of said anchoring screw;
said pressure plate having an open slit formed therein, enabling said pressure plate to be pushed in orthogonally with respect to a longitudinal direction of the shaft, for securing said pressure plate in an axial direction on said shaft.

2. The device according to claim 1, wherein a diameter of said shaft is not smaller than a diameter of said anchoring screw.

3. The device according to claim 1, wherein said shaft is formed with a circumferential groove for securing said pressure plate on said shaft.

4. The device according to claim 1, wherein said pressure plate is formed with a bore for aiding during a withdrawal thereof.

5. The device according to claim 1, wherein said pressure plate is formed with an upwardly curved edge.
